(19) United States
(12) Patent Application Publication
(10) Pub. No.: US 2021/0285062 A1
(43) Pub. Date: Sep. 16, 2021

(54) FLUORESCENCE DETECTION IN YEAST COLONIES

(71) Applicant: THE REGENTS OF THE UNIVERSITY OF CALIFORNIA, Oakland, CA (US)

(72) Inventors: Philipp Jaeger, La Jolla, CA (US); Trey Ideker, La Jolla, CA (US)

(21) Appl. No.: 16/613,931

(22) PCT Filed: May 15, 2018
(86) PCT No.: PCT/US2018/032819

§ 371 Date: Nov. 15, 2019

Related U.S. Application Data
(60) Provisional application No. 62/507,087, filed on May 16, 2017.

(51) Int. Cl. C12Q 1/6895; C12Q 1/06; C12N 1/16; C12Q 1/6897

(52) U.S. Cl. CPC C12Q 1/6895; C12Q 1/6897; C12N 1/16; C12Q 1/06

(57) ABSTRACT

Disclosed herein are improved methods for fluorescence measurements in yeast colonies.

### CROSS-REFERENCE TO RELATED APPLICATIONS

This application claims the benefit of U.S. provisional patent application 62/507,087, filed May 16, 2017; the entire contents of which are incorporated by reference herein.

### STATEMENT REGARDING FEDERALLY SPONSORED RESEARCH

This invention was made with government support under Grant Nos. R01ES014811 and R01GM084279 awarded by the National Institutes of Heath. The government has certain rights in the invention.

### BACKGROUND

Yeast is an extremely versatile eukaryotic single-cell model organism with a large selection of elegant tools available for high-throughput screening. For example, genome-wide gene deletion collections have been used very successfully to map out epistatic relationships between yeast genes under various conditions or to perform suppressor and enhancer screens to discover genetic modifiers of biological processes. To date, assays carried out with thousands of yeast mutants arrayed onto agar plates and propagated robotically represent a fast and accurate way to execute these experiments. The most commonly used phenotypic readout is colony size, a stand-in for growth rate and a very high-level, coarse abstraction of a multitude of otherwise unobservable molecular phenotypes.

### SUMMARY

Disclosed herein are methods for detecting fluorescence in a yeast colony, the method comprising: inoculating an agar plate with yeast colonies in a grid pattern; transferring the yeast colonies to a membrane; allowing the colonies to grow on the membrane; imaging the membrane at an appropriate wavelength to detect fluorescence associated with the colonies; and quantifying the fluorescence associated with at least one colony. In some embodiments, the membrane is a nitrocellulose membrane.

### BRIEF DESCRIPTION OF THE DRAWINGS

FIG. 1 depicts an imaging set up according to embodiments disclosed herein.

FIG. 2A depicts the intensity of emission/excitability/translucency of GFP, the LED light source, and the band pass filter on the camera at various wavelengths according to embodiments disclosed herein. FIGS. 2B and 2C depict the excitation (2B) and emission (2C) spectra of additional fluorescent reporters as published in Cranfill et al., Nat Methods. 13(7): 557-562,2016.

FIG. 3 depicts the arrangement of the yeast colonies, the nitrocellulose membrane, and the agar surface.

FIGS. 4A-4I depicts an exemplary image of yeast colonies using the methods disclosed herein. FIGS. 4A-4D depict images of yeast colonies on agar. FIGS. 4E-4H depict the same colonies grown on nitrocellulose membranes. “White light colony view” refers to the regular white light view of a colony tester plate consisting of sets of four colonies either fluorescent (high/low green fluorescent protein (GFP)) or not (no GFP) (see FIG. 4I for legend). “Blue light colony view” refers to the same plate imaged under blue light of approximately 460 nm. The presently disclosed method reduced the autofluorescence (the fluorescence of the “no GFP” colonies). FIGS. 4C, 4D, 4G, and 4H are higher magnification images of the colonies in FIGS. 4A, 4B, 4E, and 4F, respectively. A dramatic improvement in fluorescence and an almost complete absence of autofluorescence is seen in the colonies grown on nitrocellulose (FIGS. 4E-4H) over those grown on agar (FIGS. 4A-4D).

FIGS. 5A-5C depicts quantification of fluorescence of a set of four colonies (FIGS. 5A and 5B) and a quantification of the signal to noise ratio (FIG. 5C).

FIGS. 6A-6B depicts a representative example of millimeter sized colonies detected with the disclosed methods (FIG. 6A) and FIG. 6B depicts an example application of this technology to identify genes that encode protein subunits of the proteasome by utilizing a GFP-tagged misfolded-protein substrate.

### DETAILED DESCRIPTION

Yeast libraries are powerful genetic screening tools to understand the molecular basis of drug actions, protein-interaction networks, or gene-gene relationships. Florescent reporters and tag systems could add a multitude of potential applications to high throughput screening platforms using yeast as a model system (i.e. expression reporter, protein tags, steady-state protein levels, etc.), currently limited to colony growth alone. Current fluorescent setups suffer from low signal-to-noise ratios either prohibiting deployment of fluorescent markers altogether or require additional fluorescent markers to be used for baseline comparison.

This has led to only a small number of fluorescent high-throughput studies published, each requiring the use of slow and costly laser scanners or high-throughput microscopes.

Screening thousands of yeast colonies arrayed in a systematic fashion onto the surface of growth media containing gel matrices and assessing their growth is a staple assay for the exploration of genetic interaction networks or drug profiling. Currently, most screens only use colony size. Fluorescent reporters so far have been used only in very few limited cases due to the constraints described below. Described herein are methods to improve the signal-to-noise ratio of fluorescent yeast colony signal 10-20-fold with no additional assay time necessary (imaging time per plate <5 sec).

Existing art does not allow for the high-throughput quantification of single-channel fluorescence in yeast colonies grown on a solid substrate due to high autofluorescence, low signal intensities, slow imaging times, and high cost. The methods disclosed herein allow this quantification in an inexpensive and fast way. The disclosed methods allow the design and screening of a multitude of fluorescent reporters, e.g. enhanced GFP (EGFP), driven by specific promoters or fused to specific proteins.

The use of these novel phenotypic assays allows access to temporal, spatial, and molecular information in great detail, while still using the established and very fast high-density array-imaging pipeline. Long-term colony propagation over the course of weeks allows the identification of modifiers of telomere aging or minute-scale resolution imaging over 12 hours to recover the sequence of events following DNA damage. New assay technology allows the monitoring of the effects of gene deletions on the degradation of fluorescently labeled protein substrates that were targeted to specific cellular compartments or to assess abundance and post-translational modification status on any desired protein on a genome-wide scale. Any of these screening technologies can be used alone or in combination and they represent a unique set of tools ready to capture numerous molecular phenotypes, thus opening up an almost unlimited data trove for molecular phenomics (i.e. the highly parallel quantification of phenotypes) in yeast.

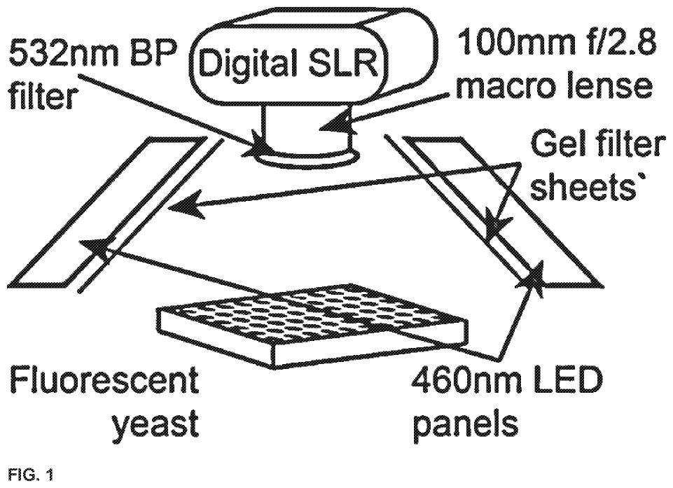

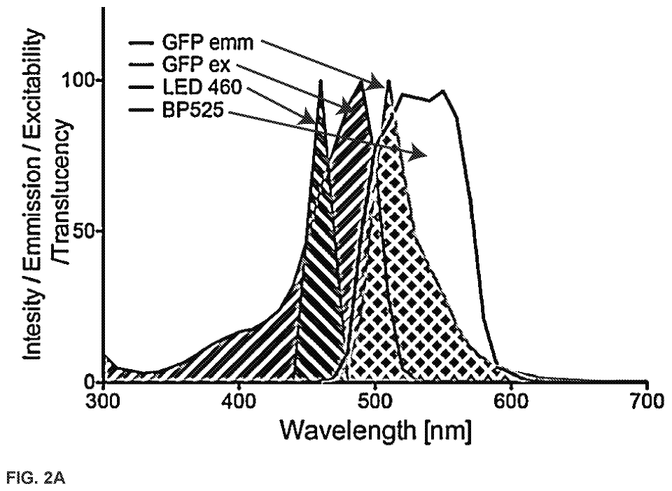

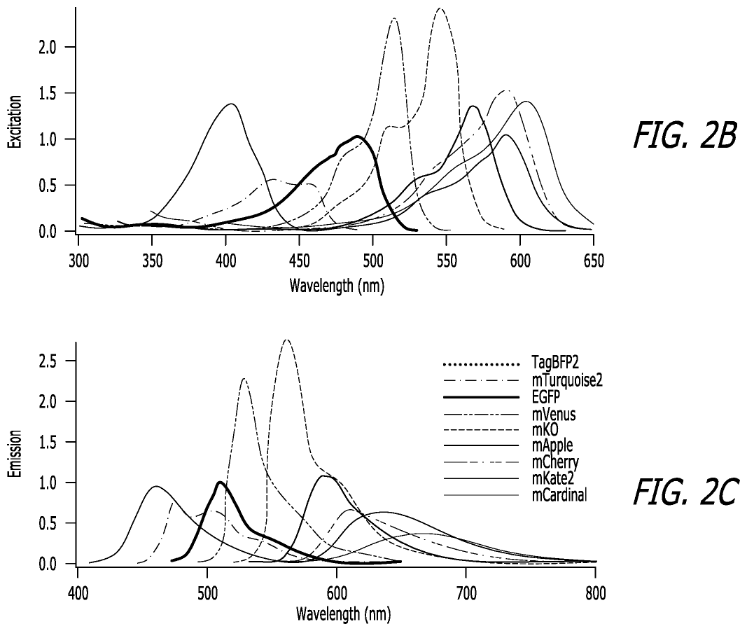

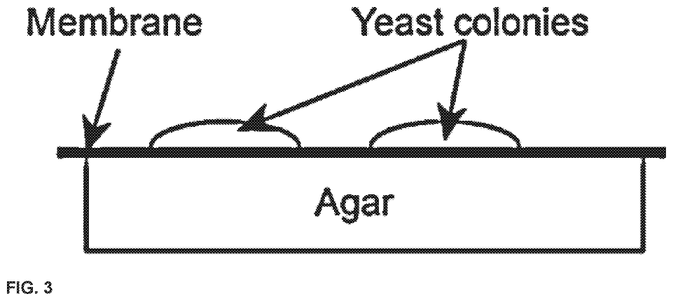

Yeast colonies are arrayed on agar in a systematic, grid pattern. In some embodiments, each colony carries specific genetic manipulations. Next, these colonies are transferred to a membrane positioned flat on an agar surface (in a non-limiting example by use a pinning robot). After growth overnight, the colonies are ready for imaging (FIG. 3). Imaging is accomplished using a system such as the one depicted in FIG. 1. A digital camera is mounted on an overhead stand and acquires a picture of the colony plate through a specific bandpass filter. Illumination is provided by two LED panels emitting light at the appropriate excitation frequency and filtered through gel filters to further improve excitation wavelength (FIG. 2). Details of the imaging system can vary; for example, other sources of excitation irradiation, in the same or other physical arrangement, can be used, so long as an even intensity of illumination is achieved across the membrane. Data from the assay membranes can be extracted through quantification with a variety of imaging systems, either analogue or digital in nature, well known to those skilled in the art of scientific imaging and data acquisition. Examples of such quantification devices capturing reflected or emitted radiation from the membrane are: analogue camera systems with film for later processing; digital camera systems with sensors that translate radiation into digital images; scanning devices such as flatbed or laser scanners; and others. If a fluorescent reporter other than GFP is used the excitation wavelength and bandpass filter will have to be chosen according to the properties of that reporter. Examples of other popular fluorescent reporters include TagBFP2, mTurquoise, mVenus, mKO, mApple, mCherry, mKate2, and mCardinal (the “m” indicating that these are monomeric proteins) (FIGS. 2B and 2C), but many others are commercially available. Using such fluorescent reporters, signals from the colonies on the membrane can be elicited and recorded over a range of wavelengths with the appropriate imaging lenses and filter systems, well known to those skilled in the art of fluorescence imaging and microscopy. In some experimental systems more than one fluorescent reporter may be used which will entail collecting an image for each reporter with appropriately matched excitation sources and filters.

Image processing and colony size and fluorescence quantification are then accomplished. After the radiation from the membrane has been captured, data are extracted by using one or more computer programs to translate the images into quantitative or qualitative data for further processing. These computer programs can be commercially available, consumer grade products (e.g., Adobe Photoshop, Canon Photo Maker, and the like), more specialized scientific programs (e.g., ImageJ, MetaMorph), or custom-made code (e.g., MatLab, Python, R code) such as the Yeast Colony Toolkit. In the Examples below a slight modification of an existing, published software application (Bean et al., PLoS One. 2014 Jan. 21; 9(1): e85177) was used.

Different membrane or membrane-like materials can be used to support yeast growth on top of nutrient containing, solid media. Suitable membranes for use in the present methods include those membranes with are biologically inert and have a pore size which allows diffusion of nutrients from the culture media but does not allow the passage of the yeast cells. In some embodiments, the membrane can be a mixed cellulose ester membrane, a cellulose acetate membrane, a coated cellulose acetate membrane, a polytetrafluoroethylene (PTFE) membrane, a nylon membrane, a polycarbonate membrane, a polyvinylidene fluoride (PVDF) membrane, or a polyamide membrane. In certain embodiments, the membrane is a nitrocellulose membrane.

The solid media contains at a minimum the nutrients required to support the desired level of growth of the supported microorganism. In some embodiments, the solid media is a standard yeast growth agar and in other embodiments, the agar incudes a chemical compound which has an effect on the yeast in order to measure a specific molecule response in the context of the yeast genome (wild type or mutated) and the chemical compound. In other embodiments solidity can be accomplished through use of other gelling agents such as noble agar, agarose, carrageenan, or phytagel. In particular, use of carrageenan or phytagel may further reduce background autofluorescence. Throughout this disclosure reference is made to agar, but it should be understood that in alternative embodiments another gelling agent is used to form the solid support.

In certain embodiments, the membrane is modified prior to use by bathing the membrane is a solution including a modifier substance. Modifier substances can be used to advantageously alter the physical properties of the membrane or to provide nutritional or other selective or inductive agents, or both. While membranes, such as nitrocellulose, can be applied to the agar dry, it is preferred to wet the membrane before applying it to the agar which help in avoiding air pockets between the membrane and the agar which would impede or prevent establishing a tight seal between the membrane and the agar, and transfer nutrients to colonies on the membrane. In some embodiments, the modifier substance is an amino acid, such as lysine or arginine, for example, to support growth of auxotrophic strains of yeast. In other embodiments, the modifier substance is an agent which changes the pH or other biophysical parameters of the membrane to modify the yeast colony shape. In other embodiments the modifier substance induces gene expression from non-constitutive promoter. In still other embodiments the modifier substance is a drug or other selective agent.

In some embodiments, the membrane is bathed in one or more of a cell culture medium, an aqueous solution, or an organic solution prior to use.

The disclosed method is useful for any strain of yeast, and for any imaging of yeast colonies having associated therewith a fluorescent tag. Yeast fluorescence colony assays are useful to detect protein expression, proliferation, yeast genotypes, yeast phenotypes, protein interactions, drug screening assays, etc.

Fluorescence is detected by a camera equipped with the appropriate filters. Any wavelength of fluorescence can be detected by the disclosed method.

Thus, described herein is a simple technology completely compatible with existing high throughput colony pinning platforms enabling the sensitive detection of colony fluorescence of thousand of colonies simultaneously with acquisition times measured in seconds at virtually no additional cost.

This technology has been used to successfully screen for modulators of promoter activity and for genetic modifiers of protein degradation.

This technology will be of great interest to both, academic researcher pursuing the mapping of molecular dependencies in yeast and to pharmaceutical research companies, using yeast as a convenient eukaryotic model organism for drug screenings.

Without being bound to any particular mechanism, according to current understanding the reduction in autofluorescence arises from physical rather than biochemical differences between yeast colonies grown on agar and those grown on a membrane. The membrane-grown colonies are visibly flatter and have more sharply defined boundaries. It is believed that these differences, including the difference curvature, alter internal reflection and other optical properties of the colony.

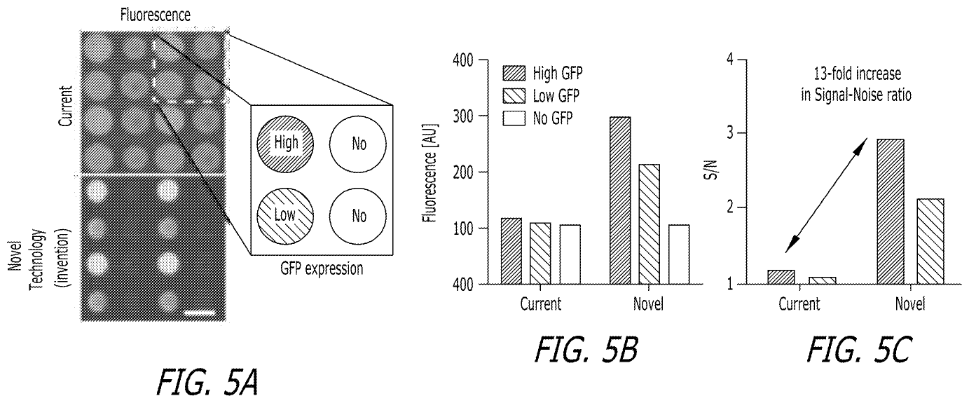

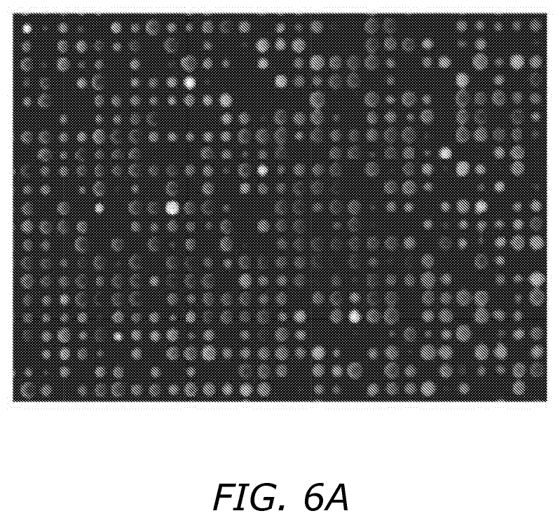

State-of-the-art technology lacks sensitivity for the high-throughput screening of thousands of yeast colonies arrayed on agar in systematic fashion. The novel technology described herein dramatically increases the fluorescence levels and increases signal to noise ratios up to 13-fold (FIGS. 5A-5C).

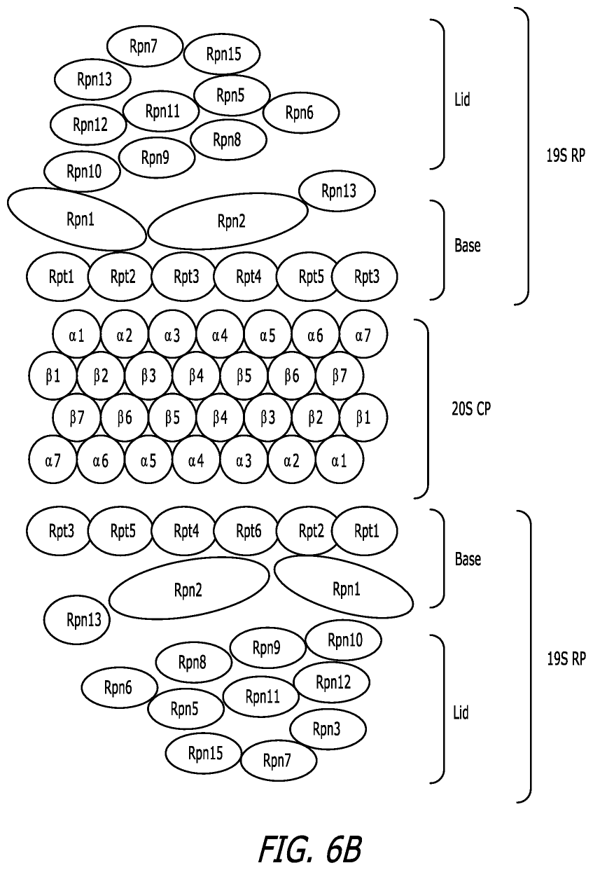

The innovative technology enables the detection of fluorescent signals reliably, even in only millimeter-sized colonies (FIG. 6A).

The presently disclosed methods allow the growth and assay of yeast colonies on the same substrate, without the need to transfer colonies from a growth substrate to an assay substrate (such as a membrane). This allows the in situ measurement of fluorescence.

### EXAMPLES

### Example 1

### Fluorescence Imaging of Yeast Colonies

Agar plates were prepared (10 mg/ml yeast extract, 20 mg/ml peptone, 0.12 mg/ml adenine, 20 mg/ml agar, glucose, and kanamycin) and allowed to rest overnight.

Yeast colonies were then grown on the agar plates at a density of 1536 colonies/plate or 6144 colonies/plate.

A nitrocellulose membrane (0.45 μm pore size) was cut to a size slightly smaller than the agar plate surface and bathed in a YPD solution (10 mg/ml yeast extract, 20 mg/ml peptone, 0.12 mg/ml adenine, glucose) for 15 min. The nitrocellulose membrane was then carefully laid down on a dry agar plate so that no bubbles formed between the membrane and the agar. Excess fluid was removed, and the membrane-agar sandwich was allowed to dry overnight to allow all the fluid to absorb into the agar.

The yeast colonies were then transferred to the nitrocellulose/agar sandwich such that the nitrocellulose surface had a colony density of 6144 colonies. The plates were then incubated at room temperature overnight.

For colony imaging, white-light images yeast colonies were acquired using a digital imaging setup with a single-lens reflex (SLR) camera (18-Mpixel Rebel T3i; Canon USA Inc.) with an 18-to-55-mm zoom lens. A white diffuser box with bilateral illumination and an overhead mount for the camera was used in a darkroom. Colony information was collected after images were normalized, spatially corrected, and quantified using a set of custom algorithms, also known as the Colony Analyzer Toolkit (githubDOTcom/brazilbean/Matlab-Colony-Analyzer-Toolkit). Digital images were cropped and assembled in Adobe Photoshop and Illustrator. Fluorescent images of yeast colonies were acquired using a custom fluorescent digital imaging setup. A SLR camera (20.2-Mpixel EOS 6D; Canon) was used with a 100-mm f/2.8 macro lens (Canon) and a green band-pass filter (BP532; Midwest Optical Systems, Inc.). A 460-nm LED panel (GreenEnergyStar) with a ¼ white diffusion filter (251; Lee Filters) for 45° bilateral illumination (205560; Kaiser Fototechnik GmbH & Co. KG,) and an overhead mount for the camera (205510; Kaiser) was used in a darkroom.

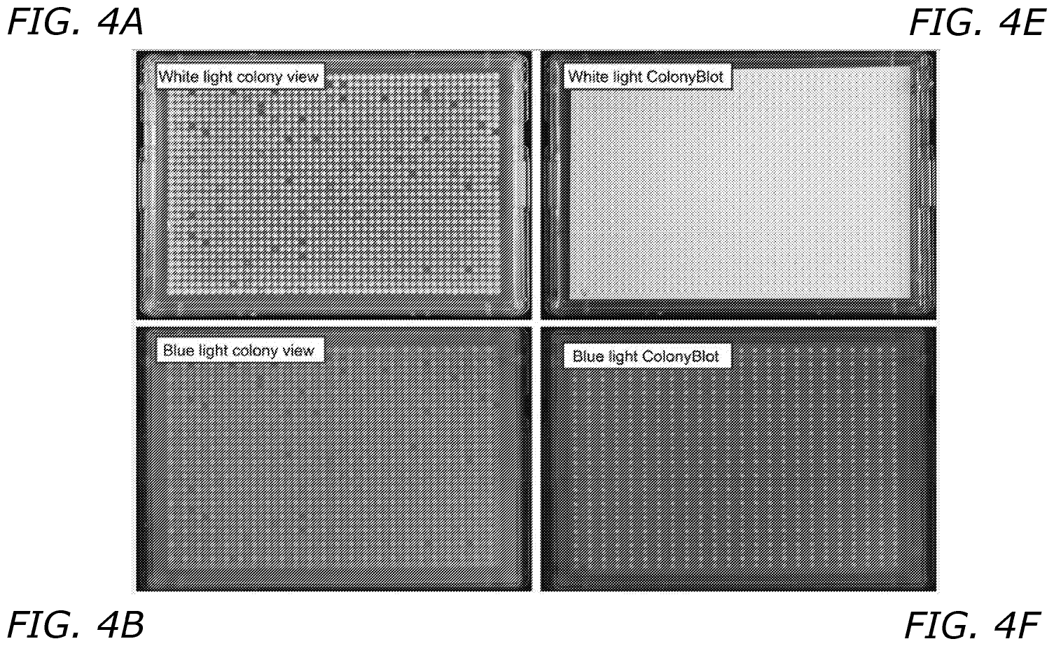

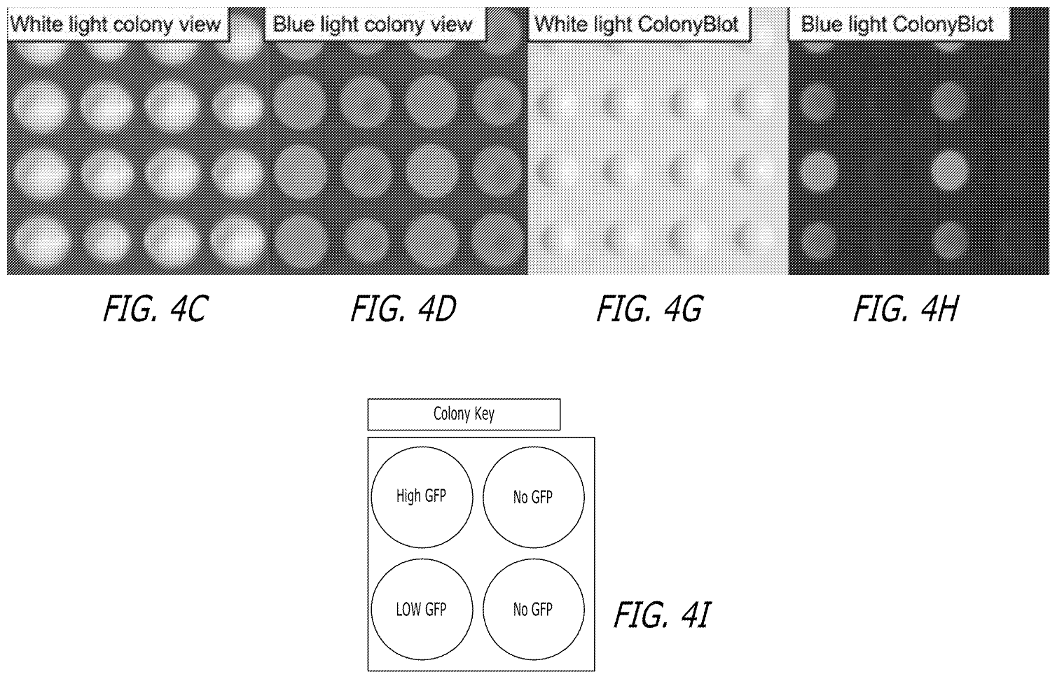

Exemplary images acquired are depicted in FIGS. 4-6A.

FIG. 4A is a white light image of yeast colonies grown directly on agar. The empty positions in the grid arise from a plate-identification, “watermarking” procedure. White light imaging is neither a necessary or typical part of the procedure but is done here for illustrative purposes. FIG. 4E is a white light image of the same colony array after transfer to and growth on a nitrocellulose membrane above agar. While referred to as a Colony blot, transfer was actually accomplished by pinning onto the membrane already laid down on the agar as described above.

FIG. 4B and FIG. 4F are images of green fluorescence produced under blue illumination of the same plates shown in FIG. 4A and FIG. 4B, respectively. As depicted in FIG. 4I the colonies are arranged in groups of four with two GFP expressing colonies to the left, a high expresser above a low expresser, and two GFP non-expressing colonies to the right. As a result of this arrangement, the even numbered columns contain GFP non-expressing colonies, which is immediately apparent in FIG. 4E. It is also discernable in FIG. 4B, but the autofluorescence from the GFP non-expressers nearly swamps out the difference; indeed there is essentially no difference in fluorescence between the low and non-expressers. FIGS. 4C and 4D, and 4G and 4H show higher magnification images of matched sections of 4A and 4B, and 4E and 4F, respectively. The different levels of fluorescence between high, low, and no GFP expression are clear for the colonies grown on the nitrocellulose membrane (4H), but are much more difficult to discern for the colonies grown directly on agar (4D).

FIG. 5 shows matched images used for quantitation and comparison of fluorescence of colonies grown directly on agar with colonies grown on a nitrocellulose membrane over agar (5A), the quantitation from representative sets (N=384) of four colonies (5B), and a comparison of signal-to-noise ratio (5C). Not only can fluorescence from high, low, and no GFP expression be more readily distinguished, but overall observed signal strength from GFP expression is increased. Additionally, the signal-to-noise ratio is increased for the membrane grown colonies by about 13-fold (more than an order of magnitude) over the colonies grown directly on agar.

FIG. 6A shows data from an experiment (similar to that in Example 3, below) revealing gene products participating in proteasomal degradation of a GFP-tagged misfolded-protein substrate. Normally this protein is degraded and there is little or no fluorescence from GFP. When a gene encoding a subunit of the proteasome (see FIG. 6B) is absent or proteasome activity is otherwise impaired, fluorescence due to GFP expression survives. The more proteasomal activity is impaired the greater the fluorescence.

### Example 2

### Genome-Wide Assessment of Glucose Repression Pathway

As a specific and biologically relevant test of the disclosed assay technology to utilize systematic gene-to-phenotype arrays (SGPAs) to comprehensively map the genetic landscape driving molecular phenotypes of interest. By this approach, a complete yeast genetic mutant array is crossed with fluorescent reporters and imaged on membranes at high density and contrast. Importantly, SGPA enables quantification of phenotypes that are not readily detectable in ordinary genetic analysis of cell fitness.

We explored a fundamental cellular process: the inducible, tightly controlled GAL1 promoter (pGAL1), a classic readout of the so-called glucose repression pathway. By deploying multiple copies of a pGAL1 fluorescent transcriptional probe per cell, we quantified promoter activation and repression under induced and repressed conditions, respectively, across approximately 6,000 mutant yeast strains. In this context, we found that SGPA enables a broadly useful and sensitive approach to gene discovery, particularly when applied to inherently weak phenotypes such as leaky promoter activity. We identified the highly conserved Mediator complex as a crucial element in transcriptional control from the GAL1 promoter. Dynamic module changes in Mediator play a central role in controlling eukaryotic transcription. SGPA uncovered a role for the CDK8/kinase module in regulating both promoter repression and induction, depending on environmental context, and identified module interfaces involved in complex function.

Strains from the YKO and DAmP collections (GE Dharmacon, Lafayette, Colo.) were grown on YPAD medium with 100 mg/ml G418 at 96 colony density and then manually re-arrayed to remove blank spaces, non-growing strains, and duplicates, resulting in the SPOCK collection (single-plate ORF (open reading frame) compendium kit). A complete strain list and location map can be found in Jaeger et al., Molecular Cell 69, 321-333, 2018. The 96 well plates were then re-pinned and condensed to 6144 colony density using the Rotor HAD (Singer Instruments, Taunton, UK). Mating with the pGAL1 query strains and selection were performed using standard E-MAP procedures, except that all incubation steps took place over-night at room temperature to avoid overgrowth. After double mutant selection, strains were pinned onto agar (for fitness measurements) or onto 0.45 μm nitrocellulose membrane (BioRad, Hercules, Calif.; for fluorescence measurements). The membrane was pre-wetted with selection media and rolled onto the agar surface to avoid bubble formation.

Media preparation, genetic and molecular biology techniques were carried out using standard methods: Yeast strains were cultured using yeast extract/peptone/dextrose (YPD) at 30° C. Majority of the deletion strains used were in the BY4741 (MATa ura3Δ0 leu2Δ0 his3Δ1 met15Δ0) background derived from the Resgen Deletion Collection (GE Dharmacon) except the Y7092 query strain. The Y7092 strains carried the respective insertions for each of the generated screens using standard LiOAc protocols for transformation:
ade2Δ::URA3-ADE2
ade2Δ::URA3-ADE2-pTDH3-ΔssCPY*
ade2Δ::URA3-ADE2-pTDH3-ΔssCPY-GFP
ade2Δ::URA3-ADE2-pTDH3-ΔssCPY-NES-GFP
ade2Δ::URA3-ADE2-pTDH3-ΔssCPY-GFP san1Δ::cNAT

The plasmid cytoplasmic Carboxypeptidase-Y protein DssCPY*-GFP (pRH2081) was provided by D. Wolf (University of Stuttgart, Stuttgart, Germany). tGND1 (pRH2476), and DssCPY*-GFP-NES (pRH2557) were developed in-house. Plasmids were heat-shock transformed into competent E. coli (DH5a), recovered using standard Mini-Prep protocols (Promega), and re-transformed into yeast cells using standard procedures. Competent colonies were selected with the appropriate selection conditions.

Bacto agar (#214040, BD Biosciences, San Jose/Calif.) was used as the gelling agent. Supplemental reagents and media were Bacto yeast extract (#212720, BD Biosciences), Bacto peptone (#211820, BD Biosciences), Difco Dextrose/Glucose (#215520, BD Biosciences), Difco Yeast nitrogen base without amino acids (#291920, BD Biosciences) and Difco Yeast nitrogen base without amino acids and ammonium sulfate (#233520, BD Biosciences). In case of the galactose experiments, glucose (2%) was replaced with an equal percentage galactose (2%). Synthetic complete (SC) or SC-dropout media were prepared following standard procedures using amino acids from Sigma-Aldrich. If indicated, selective pressure was maintained using geneticin (G418, KSE Scientific, Durham/N.C.), S-(2-Aminoethyl)-L-cysteine hydrochloride (S-AEC, A2636, Sigma-Aldrich), or L-(+)-(S)-Canavanine (Can, C9758, Sigma-Aldrich) at the indicated concentrations. Gelling, supplemental, and media reagents were mixed in ddH2O and autoclaved for 15 min at 121° C. before use; selective drugs were added after the liquid gel solution cooled to below 60° C. in a water bath.

### Example 3

### Genome-Wide Assessment of Protein Quality Control

Utilizing the same general procedures as described in Example 2, except mating to CPY query strains of yeast, we sought to genetically dissect molecular phenotypes related to carboxypeptidase Y (CPY), a well-established substrate for the study of protein quality control (PQC) pathways. A permanently misfolded state in the normal CPY protein is induced by a single amino acid substitution denoted CPY*. Subsequent removal of the endoplasmic reticulum import signal sequence (ss) and addition of GFP result in the model cytoplasmic misfolded protein ΔssCPY*-GFP. Normally, this misfolded protein is rapidly degraded by PQC machinery, whereas disturbances in PQC are identified by accumulation of ΔssCPY*-GFP. Specifically, ΔssCPY*-GFP is marked for degradation by the San1p and Ubr1p ubiquitin ligases in the nucleus versus cytosol, respectively, while deubiquitinating enzymes like Ubp3p promote its stabilization.

SGPA, incorporating an embodiment of the herein disclosed technology, was used to comprehensively evaluate the effect of yeast gene mutations on levels of ΔssCPY*-GFP integrated as a single copy at the ADE2 locus. To eliminate genes that have general effects on GFP expression or brightness rather than roles in PQC, we assessed the differential fluorescence between each mutant expressing either misfolded ΔssCPY*-GFP or GFP alone. In a total of 274 gene deletion mutants, we observed significant changes in GFP colony fluorescence relative to control.

As a first validation of these results, we scored the extent to which the SGPA gene set recovered known components of PQC, including the established ubiquitinating/deubiquitinating enzymes and the proteasome complex. The approach recovered mutant strains for both the ubiquitin ligases (san1Δ and ubr1Δ) and the deubiquitinating enzyme (ubp3Δ), which played opposing roles on the test substrate: loss of the known ligases resulted in elevated GFP levels, while loss of the deubiquitinating enzyme resulted in decreased GFP levels and altered degradation kinetics (pdr5Δ serves as wild-type control). SGPA also recovered 70% (21/30) of essential proteasome complex members based on a strong increase in GFP fluorescence in the hypomorphic mutant strains. In contrast, we noted very little change in cellular fitness due to deletion of any of these genes, demonstrating the difficulty in studying a basic biological process such as PQC with a simple assay based only on cellular growth.

To assess the robustness of these results to defined changes in subcellular location of the misfolded protein. Accordingly, we performed two independent follow-up screens with well-characterized substrate derivatives: first, we used a modified fluorescent substrate predominantly localized in the cytosol (ΔssCPY*-GFP-NES, ΔssCPY*-GFP with a nuclear export signal). Second, we deleted the nuclear ubiquitin ligase SAN1 across all mutants, which is involved in proteasome-dependent degradation of aberrant nuclear proteins (ΔssCPY*-GFP san1Δ). All three screens yielded highly overlapping hits (p<<10⁻⁸), indicating that misfolded CPY identification and degradation employ similar mechanisms independent of subcellular localization. Due to this overall similarity, we took the union of all three screens to create a unified dataset of 556 mutants with either significantly increased or decreased fluorescence compared to wild-type. A total of 312 versus 244 mutants were associated with decreased or increased ΔssCPY* fluorescence, respectively.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method to detect fluorescence in a yeast colony comprising:
inoculating an agar plate with yeast colonies in a grid pattern;
transferring the yeast colonies to a membrane;
allowing the colonies to grow on the membrane;
imaging the membrane at an appropriate wavelength to detect fluorescence associated with the colonies; and
quantifying the fluorescence associated with at least one colony.

2. The method of claim 1, wherein the membrane is a nitrocellulose membrane.

3. The method of claim 1, wherein the wavelength of the detected fluorescence is 532 nm.

4. The method of claim 1, wherein the fluorescence associated with the colonies is from green florescent protein.

5. The method of claim 1, wherein the grid has a density of at least 1536 yeast colonies/plate.

6. The method of claim 5, wherein the grid has a density of at least 6144 yeast colonies/plate.

7. The method of claim 1, further comprising allowing further growth and re-imaging the membrane at a later point in time.

8. The method of claim 1, wherein at least one yeast colony comprises a fluorescent reporter protein coding sequence is operably linked to a promoter, wherein promoter activity is to be assayed.

9. The method of claim 1, wherein at least one yeast colony comprises a fluorescent reporter protein coding sequence fused to a protein coding sequence, wherein expression or degradation of the protein is to be assayed

10. The method of claim 8, wherein the fluorescent reporter protein comprises green fluorescent protein.

11. The method of claim 1, wherein the fluorescence is associated with expression of a gene.

12. The method of claim 1, wherein the fluorescence is associated with proliferation of the yeast.

13. The method of claim 1, wherein the fluorescence is associated with a genotype.

14. The method of claim 1, wherein the fluorescence is associated with a phenotype.

15. The method of claim 1, wherein the fluorescence is associated with protein interaction.

16. The method of claim 1, wherein the fluorescence is associated with sensitivity or resistance to a drug.

17. The method of claim 9, wherein the fluorescent reporter protein comprises green fluorescent protein.
